(19) United States
(12) Patent Application Publication
(10) Pub. No.: US 2005/0033388 A1
(43) Pub. Date: Feb. 10, 2005

(54) Medical laser treatment device

(76) Inventors: Wilhelm Brugger, Burmoos (AT); Anton Kasenbacher, Traunstein (DE); Mark Niemz, Sinzheim (DE); Thorsten Bauer, Hannover (DE)

Correspondence Address: Leonard J Santisi;Frommer Lawrence & Haug, Suite 850, 4660 La Jolla Village Drive, San Diego, CA, 92122, US

(21) Appl. No.: 10/398,073

(22) PCT Filed: Feb. 22, 2001
(86) PCT No.: PCT/EP02/01906

(30) Foreign Application Priority Data
Feb. 22, 2001 (DE) 10108655.5

(51) Int. Cl. A61N 1/00

(52) U.S. Cl. 607/89

(57) ABSTRACT

A medical laser treatment appliance which can be switched between a continuous wave (cw) operating mode and a pulsed operating mode and has at least one first laser light source for producing a cw laser beam, with the laser treatment appliance furthermore having a second laser light source for producing a pulsed laser beam, which is pumped by at least one first laser light sources, and the cw laser beam from at least one of first laser light sources is used for treatment in the cw operating mode, and the pulsed laser beam from the second laser light source is used for treatment in the pulsed operating mode, with the laser treatment appliance being designed such that the laser light source is also pumped continuously in the cw operating mode by at least one of the first laser light sources.

The present invention relates to a medical laser treatment appliance having a laser light source, in particular a dentistry laser treatment appliance.

### BACKGROUND

One such laser treatment appliance is known, for example, from DE 195 33 348 A1. In addition to the laser light source, such an appliance preferably has control elements, control apparatuses, apparatuses for carrying laser light, such as glass fiber cables or waveguides, as well as a hand piece, which forms a treatment instrument. Furthermore such laser treatment appliances may have power supply elements and displays or indicators, such as monitors or lamps, which are normally arranged or provided in housing elements.

Medical laser treatment appliances are used for various medical purposes and in different embodiments. Depending on the purpose of use, the laser treatment appliances have to have different specifications, particularly with regard to the characteristic of the laser light which is emitted from them for medical treatment.

For comprehensive use of medical laser treatment appliances for different applications, the person carrying out the treatment is thus forced to use different laser treatment appliances, in particular, on the one hand, a laser treatment appliance which can emit cw (continuous wave) laser light, and, on the other hand, a laser treatment appliance which can emit pulsed laser light. However, on the one hand, it is very costly to purchase a number of laser treatment appliances and, on the other hand, the need or wish to use different laser treatment appliances in some cases during the treatment is highly inconvenient and, in some cases, is feasible only with difficulty, if at all, in the given conditions, in particular in the given time period.

Furthermore, DE 198 44 719 A 1 discloses a laser treatment apparatus, which comprises a solid state laser for producing a laser beam and an excitation light source which excites the solid state laser so that it emits continuous laser light, cw laser light. The appliance which is disclosed in DE 198 44 719 A1 furthermore comprises two different, mutually independent optical systems and a system for switching the optical path of the laser light, by means of which the cw laser light which is produced by a solid state laser is passed into one of the optical systems or into the other optical system, with this switching system being in the form of a pivoting mirror which is moved into the beam path of the continuous laser beam produced by the solid body, in order to steer the laser light into a first optical system, or is moved out of the path of the laser beam in order to allow the laser light to pass into a second optical system, without being deflected.

A system such as this has the advantage that it is relatively complex and is susceptible to defects, especially owing to the need to move the pivoting mirror mechanically. Furthermore, the switching process is time-consuming and only laser light from a single laser light source can be accessed, so that the variation options and options for use of such a system are restricted.

The object of the present invention is thus to provide a medical laser treatment appliance which can be used in a more variable manner, in which case it is possible to use different laser light sources and, furthermore, it is possible to switch quickly and without any susceptibility to defects between individual operating modes of the appliance.

This object is achieved by a medical laser treatment appliance having the particularly advantageous embodiments of the medical laser treatment appliance according to the invention.

### SUMMARY OF THE INVENTION

According to the invention, the laser treatment appliance, which is designed to be switchable between a cw operating mode and a pulsed operating mode, comprises at least one first laser light source for producing a cw laser beam, and a second laser light source for producing a pulsed laser beam, with the second laser light source being pumped or excited by at least one first laser light source.

According to the invention, when the laser treatment appliance is in the cw operating mode, cw laser beam from at least one of the at least one first laser light sources is used for treatment or diagnoses while, in the pulsed operating mode, the pulsed laser beam from the second laser light source is used for treatment. Furthermore, according to the invention, the laser treatment appliance is designed such that the second laser light source is pumped continuously by at least one of the first laser light sources, even when the laser treatment appliance is in the cw operating mode. This means that at least a specific proportion of the laser light power from at least one first laser light source is used continuously to pump or to excite the second laser light source, so that this component is not available per se for the laser treatment with a cw laser beam, but the second laser light source is always in the excited state.

The laser treatment appliance according to the invention has, in particular, the advantage that two different laser systems can be used for producing a cw laser beam on the one hand and a pulsed laser beam on the other hand in a single appliance, thus considerably increasing the band width of the possible treatment and the corresponding variability, with the provision of a second laser light source in particular making it possible to provide a very high-energy pulsed laser beam, which would not be feasible if cw laser light were simply converted to pulsed laser light, for example by means of a quality switch or the like.

Since, furthermore and according to the invention, the second laser light source is excited or pumped continuously, and is thus in a continuous operating state, it is possible to switch from a cw operating mode to a pulsed operating mode without any time delay, since there is no need to “start up” the pulsed laser light source. At this point, it should be noted that the “starting up” of a pulsed laser light source generally requires at least 30 seconds, depending on the system, and in some circumstances more than one minute may pass before the pulsed laser light source is running in a stable manner, so that a person carrying out the treatment experiences long waiting times for switching from a cw operating mode to a pulsed operating mode, which would lead to unacceptable delays, especially when the operating modes are changed frequently, as is very often desirable especially in the field of dentistry.

In the cw operating mode the invention provides in a preferred manner for only a relatively small proportion of the power from at least one first light source (in comparison to the pulsed operating mode) still to be used as pump radiation for the second laser light source, so that although the second laser light source has less pump power available, this reduction in the power is, however, chosen such that the amplifier does not break down during pulsed operation, and it is possible to return to normal operation with full pump power very quickly.

In another embodiment, it is also possible for a constant pump power to always be available both in the cw operating mode and in the pulsed operating mode. This may be achieved, on the one hand, by a fixed proportion of the power produced by at least one first light source always being output as pump radiation while, on the other hand, this may be achieved by providing a number of first light sources, for example a number of laser diodes, with at least one of the first laser light sources, preferably the laser diodes, being used exclusively for pumping the second laser light source, while further first laser light sources are used for producing the cw laser beam for treatment.

In one embodiment, in which only one first laser light source is used, or a number of first laser light sources, for example laser diodes, are used in parallel and a portion of the laser power from the cw laser beam that is produced is output. A beam splitter is used as the output apparatus, in particular a beam splitter which is dependent on the polarization direction of the incident light. The polarization of the laser light which arrives at the beam splitter is produced by at least one first laser light source preferably being controllable by means of an upstream element for controlling the polarization direction. One such element may be, for example, a half-wave plate which can be rotated and is placed in the beam path of the cw laser light which is produced by the at least one first laser light source.

Solid state lasers, especially laser diodes, are particularly suitable for use as the first laser light source, while wafer lasers are particularly suitable for use as the second laser light source. A wafer laser has the advantage that the crystal which is used for the wafer laser can be cooled very effectively from virtually all sides. Since the diameter of the pump beam is generally very much larger than the thickness of the doped crystal wafer that is used, this greatly suppresses the formation of a so-called “thermal lens”, that is to say this avoids the possibility of different heating of the crystal resulting in changes, in particular distortion of the crystal which can lead to undesirable reflections, owing to the fact that the pump beam has a different intensity distribution, and generally has a Gaussian distribution.

Thus, in comparison to other laser systems, a wafer laser is also less susceptible to changes in the pump power, and its power is scalable while maintaining a constant good beam quality, which is particularly important for the laser treatment appliance according to the invention since here, depending on the mode, the second laser light source is pumped with a different power level when, for example, a greater proportion of cw laser radiation from at least one first light source is output for treatment in a cw operating mode and only a smaller proportion of this power is used for further continuous pumping or excitation of the second laser light source, in this case the wafer laser.

Depending on the desired treatment method, the cw laser light which is produced by at least one first light source and/or the cw laser light which is produced by the second laser light source and/or the pulsed laser light can then be shielded, for example by a shutter, so that the treatment is carried out either exclusively with a pulsed laser beam, exclusively with a cw laser beam, or else in parallel with both laser beams. It is, of course, also possible to shield all the laser radiation in order to interrupt the treatment, without having to switch off the laser light sources themselves.

Depending on the choice of the operating mode, the medical laser treatment appliance can thus be used for different medical purposes. This is a result of the interaction of the laser light with the material to be treated, generally biological material and in particular tissue, which, in addition to the nature of the tissue to be treated, the input wavelength and wave power, is also dependent on the chosen pulse duration and on whether pulsed radiation or continuous radiation according to the invention is being used, and what energy levels are emitted and transmitted continuously or per pulse.

The medical laser treatment appliance according to the invention thus provides a “multi-use” apparatus, which can be used for a wide range of treatment and diagnosis methods which, on the one hand, is considerably more cost-effective than the use of a number of laser treatment appliances while, on the other hand, the operability and convenience are considerably increased, since the person carrying out the treatment, generally a doctor, can carry out different treatments or treatment steps quickly and conveniently with one appliance, simply by switching between the operating modes and without changing the appliance.

In one preferred embodiment, the medical laser treatment appliance according to the invention has at least two pulsed operating modes, which have different laser pulse durations and/or different pulse repetition frequencies of the emitted laser light.

When the medical laser treatment appliance according to the invention is in the cw operating mode, this results in particular in a thermal interaction between the emitted laser light and the tissue. The medical laser treatment appliance in the cw operating mode is thus particularly suitable for coagulation and for photodynamic therapy (PDT).

The medical laser treatment appliance is preferably furthermore provided with a control apparatus for controlling the laser light power, with power levels of up to 3 watts, and preferably about 2 watts, preferably being used for coagulation in the cw operating mode of the laser treatment appliance.

The pulsed operating mode or modes of the medical laser treatment appliance according to the invention are preferably used for ablation in particular of hard tissue such as organic hard tissue, for example tooth material and in particular carious tooth material. One of the operating modes of the medical laser treatment appliance preferably has pulse durations which are shorter than 1 ps. Pulse durations in the fs range are particularly preferable for ablation, in particular in the range from 1 to 1000 fs, preferably in a range of 700 fs and in particular from 5 to 500 fs.

In these operating modes, the medical laser treatment appliance according to the invention leads essentially to a nonthermal interaction with the hard tissue to be treated, so that the ablation process is essentially carried out without heating the surrounding tissue, in particular the surrounding hard tissue.

A further preferred operating mode of the medical laser treatment appliance according to the invention has pulse lengths in the picosecond range, in particular from 20 ps to 500 ps. A preferred pulse length for this operating mode is about 100 ps.

This operating mode is particularly suitable for sealing hard tissue surfaces.

In a further preferred operating mode of the medical laser treatment appliance according to the invention, laser pulses with a pulse duration of 1 nanosecond or more are produced. In a similar way to that in the cw operating mode, these very long pulse durations are especially suitable for coagulation of tissue.

A combination of at least two of the abovementioned operating modes and/or further operating modes thus allows the medical laser treatment appliance to be used in a versatile manner. The person carrying out the treatment, in particular the doctor, is thus able to achieve other medical effects simply by switching the operating mode, without changing the appliance and without any time delay. Thus, by way of example for a dentist, it is possible to switch during the ablation of tooth material from a pulsed operating mode to a cw operating mode when bleeding occurs, so that the bleeding can be stopped by means of coagulation without any delay. It is then possible to change back to the ablation process once again without any delay and without the dentist needing to divert his attention away from his patient in order to change the treatment appliance in which case, especially when switching to the pulsed operating mode, the capability to switch with virtually no delay as made possible by the laser treatment system according to the invention is of major importance. The patient can thus be treated more quickly, with fewer problems, better and more cost-effectively.

The medical laser treatment appliance preferably has a laser light source which emits laser light at a wavelength of λ=750 nm to λ=1100 nm. This wavelength is particularly preferable since the absorption of biological tissue in this wavelength range is relatively low so that, especially in the operating modes in which there is thermal interaction with the tissue, a high penetration depth and thus uniform and effective treatment is obtained.

This range is thus particularly suitable for tissue coagulation, while high absorption is advantageous for ablation. High absorption in dentine is achieved especially with short-pulse lasers, owing to the high intensity, even at it wavelength of 780 nm. Furthermore, higher absorption is achieved even with lower intensities especially in the UV band and at wavelengths of <500 nm, in particular at 390 nm or at 248 nm (ArF laser).

A Yb:KGW laser, a Yb:KYW laser or a Yb:YAG laser with an emitted wavelength of 1030 nm is preferably used as the second laser light source. Furthermore, it is also possible to use an Nd:YAG laser emitting a wavelength of 1064 nm or an Nd:YLF Laser emitting a wavelength of 1053 nm. Diode lasers in particular are also suitable as the first laser light source, operating in a wavelength range from 805 nm to 980 nm, for example with an emitted wavelength of 809 nm, 940 nm or 980 nm.

Typical parameters for advantageous lasers are shown in the following table:

| | Laser | | | |
|---|---|---|---|---|
| | Nd: YAG | Er: YAG | Diode laser | Yb: KGW |
| Power | LOW-5 kW | <200 W | IW −> 100 W | <100 W |
| Wavelength | 1064 nm | 2940 nm | 808, 1500 nm | approx. |
| | | | | 130 um |
| Pulse duration | approx. | approx. | cw | <lps |
| | 10 ns-cw | 10 ns-ms | | |

In a further embodiment, the medical laser treatment appliance also has a scanning apparatus for producing a scanning pattern on the surface to be treated or in the area to be treated. The scanning apparatus preferably has controllable mirrors which deflect the laser beam that is produced such that it passes, preferably cyclically, through a predetermined, controllable x-y deflection movement.

The scanning apparatus is preferably controllable by means of a computer, so that the scanning pattern, the scanning area and the scanning rate can be matched to the desired treatment aims. The scanning rate, which is quoted in distance per unit time as the units, is preferably in ranges from 10 to 1000 mm/s, in particular 100 to 500 mm/s, with a particularly preferred value being about 200 mm/s. The scanning frequency, which should be understood to mean the complete cycle of the scanning pattern per unit time, depends on the scanning rate on the one hand and on the scanning pattern and scanning area on the other hand, and is preferably in a range from 1 Hz to 1 kHz, especially 1 to 100 Hz, with a particularly preferred value being approximately 10 Hz.

The treatment success depends not only on the chosen wavelength and the pulse duration but also on the pulse repetition frequency, which is preferably likewise adjustable as a function of the treatment mode.

It has been found that ablation is required and is particularly effective especially with a short laser pulse duration and a high pulse repetition frequency. Hard tissue surfaces can be sealed particularly effectively using medium pulse lengths in the range from 20 ps to 500 ps and also lower pulse repetition frequencies, in particular pulse repetition frequencies which are less than 1 kHz. Coagulation is best produced with very long pulse durations and preferably with cw radiation.

In one preferred embodiment, a control apparatus is therefore provided which, in combination with the operating mode of the laser treatment appliance, also controls further characteristic emission or treatment characteristics of the laser treatment appliance, in particular the scanning rate or scanning frequency and the pulse repetition frequency. These may be preset automatically, or else may be manually controllable.

The medical laser treatment appliance can thus preferably be operated in an ablation mode in which both a short pulse duration of t<1 ps and a high pulse repetition frequency in a range from 1 kHz to 50 kHz, in particular about 30 kHz, are provided.

In a further mode, the sealing mode, the medical laser treatment appliance and/or the control apparatus are/is preferably designed such that the medical laser treatment appliance produces pulses with a pulse duration from 20 ps to about 500 ps with a pulse repetition frequency for treatment which is less than 1 kHz.

In a further “coagulation mode”, the medical laser treatment appliance and/or the control device are/is preferably designed such that the medical laser treatment appliance emits laser light in a cw mode for treatment, with the scanner apparatus being switched off, so that an essentially stationary or fixed-position laser beam is produced in the laser treatment appliance.

In one preferred embodiment of the medical laser treatment appliance, a switching apparatus is also provided, which either allows switching between the combination modes mentioned above, which predetermine both the operating modes (in particular the pulse duration and/or the cw mode) and the scanning frequency of the scanner and the pulse repetition frequency, or else allow separate switching between the operating modes and other characteristic operating data of the medical laser treatment appliance. In particular, the switching apparatus also has elements for preferably continuously variable power control of the medical laser treatment appliance.

In a further preferred embodiment of the medical laser treatment appliance, a visible auxiliary laser beam is also provided, which can be produced both before and during the treatment in order to make it easier for the person carrying out the treatment to carry out the treatment, especially in the case of a non-contact treatment. The medical laser treatment appliance according to the invention may, for example, for this purpose have an apparatus for frequency doubling, with a portion of the cw laser beam which is produced by at least one first laser light source preferably being output and being used as the auxiliary laser beam. However, it is also possible to use an additional laser light source, in particular a laser diode, which emits in the visible wavelength band. It is particularly preferable for this auxiliary laser light source or the apparatuses for producing an auxiliary laser light beam to indicate the operating state of the medical laser treatment appliance. This may be done, for example, by the laser light of the auxiliary laser light beam blinking when no treatment beam is being produced, and radiated continuously when treatment laser light is being emitted, irrespective of whether this is continuous or pulsed. It is, of course, also possible to vary the wavelength of the emitted auxiliary laser light depending on the operating state of the laser treatment appliance. Accoustic indicating apparatuses may also be provided in order to indicate the operating state, either in an isolated form or else in combination with the apparatuses mentioned above.

### DESCRIPTION OF THE DRAWINGS

This and further advantages and features of the medical laser treatment appliance according to the invention will become clear with reference to the following figure. In the schematic illustrations:

FIG. 1 shows an overview illustration of elements of one embodiment of a medical laser treatment appliance;

FIG. 2 shows an illustration of an apparatus for producing an auxiliary laser beam according to one embodiment of a medical laser treatment appliance according to the invention;

FIG. 3 shows the design of a number of elements of one embodiment of a medical laser treatment appliance;

FIG. 4 shows parameters for preferred operating states of a medical laser treatment appliance according to the invention;

FIG. 5 shows a design for a stretcher, as may be used in one embodiment of the laser treatment appliance according to the invention;

FIG. 6 shows a design for a compressor, as may be used in one embodiment of the laser treatment appliance according to the invention; and

FIG. 7 shows, schematically, a cross section through a number of elements of a wafer laser system.

### DESCRIPTION OF PREFERRED EMBODIMENTS

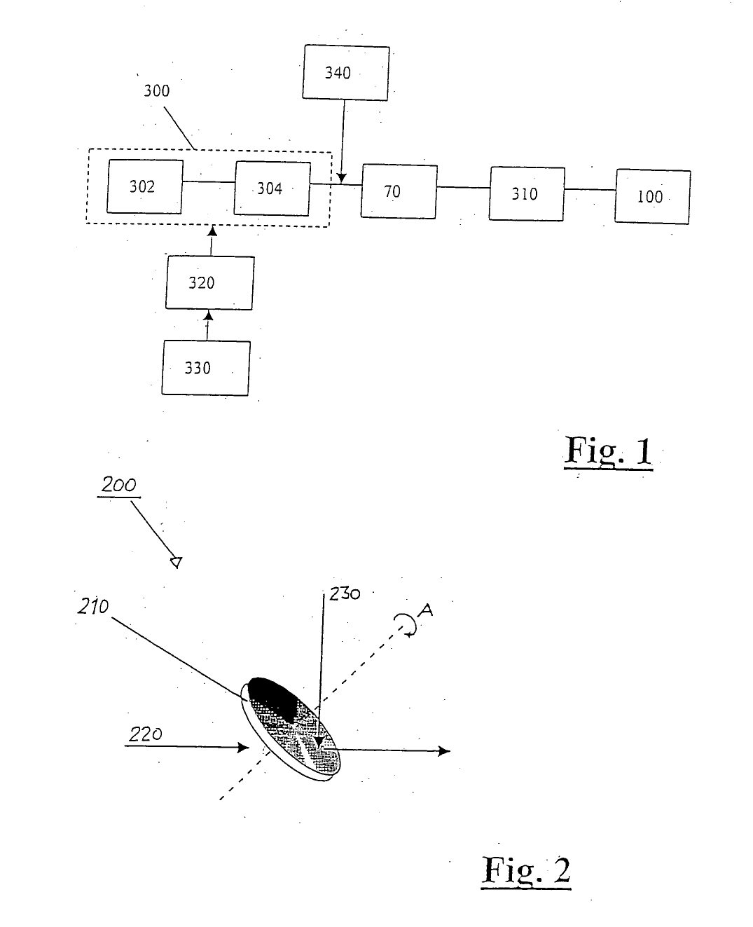

FIG. 1 shows, schematically and in the form of an overview illustration, a number of elements of one embodiment of a laser treatment appliance. The medical laser treatment appliance in this embodiment has laser light sources 302, which have a first laser light source comprising a number of laser diodes, and a wafer laser as the second laser light source, and an apparatus 304 for controlling the operating modes, which may also be assembled to form a combined laser unit 300.

The laser unit 300 or the laser light sources 302 and the apparatus 304 for controlling the operating modes are controlled via a control apparatus 320, which can in turn be operated by a switching apparatus 330, which can be operated by the person to be operated, generally a doctor or a technician.

The medical laser treatment appliance shown schematically in FIG. 1 also has an apparatus 340 for inputting an auxiliary laser beam, although it should be mentioned that this apparatus is purely optional.

The treatment laser beam and possibly the auxiliary laser beam are passed on via optical elements, with only one optical transmission apparatus 70 being illustrated here, in symbolic form.

The embodiment of the medical laser treatment apparatus illustrated here furthermore also has a controllable scanning apparatus 310, so that the laser beam can be given a scanning movement.

The laser light, to which a scanning movement can be applied, is passed to a handpiece 100, which is used for handling by the person carrying out the treatment. At this point, it should also be mentioned that the scanning apparatus 310 may also in all cases be integrated in the handpiece 100.

The design illustrated in FIG. 1 is intended merely to illustrate the schematic structure and, of course, the sequence of the illustrated elements may be interchanged, individual elements may be omitted or additional elements may be added, without having to depart from the scope of the invention described by the claims.

FIG. 2 shows, schematically, an apparatus 200 for inputting an auxiliary laser beam, as can also be used in the embodiment shown in FIG. 1 (see 340 in FIG. 1). A filter 210 is in this case used as a dichromatic beam splitter, which is essentially transparent to a treatment laser light 220. The auxiliary laser beam 230 which, for example, can be produced by a laser diode and is in the visible wavelength band, is reflected by the filter 210, so that the treatment laser beam 220 can be superimposed on it. In the embodiment illustrated in FIG. 2, the reflection of the auxiliary laser beam 230 is ensured by a reflection coating on the filter 210,

The intensity of the treatment laser beam 220 can be varied continuously by rotating the filter 210 about its axis, as indicated by the arrow A. The intensity of the auxiliary laser beam is essentially uninfluenced by the rotation of the filter 210.

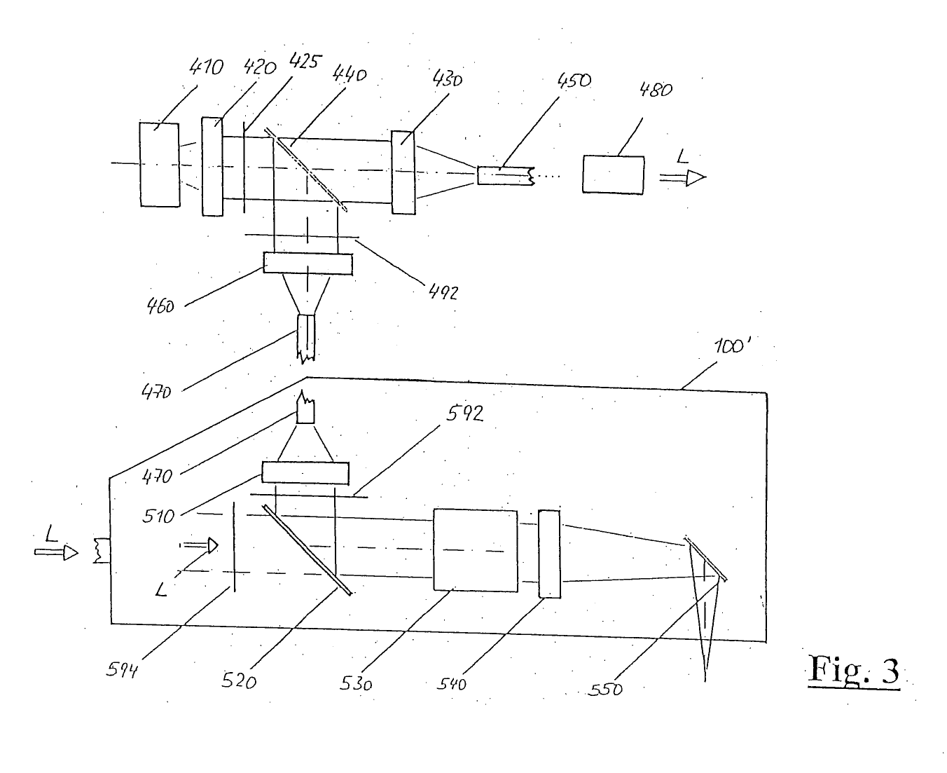

FIG. 3 shows, schematically, elements of a further embodiment of a medical laser treatment appliance according to the invention.

The laser light source illustrated in the upper part of FIG. 3 has a laser diode 410 as the first laser light source and a wafer laser 480 as the second laser light source. The continuous wave laser light emitted by the laser diode 410 is aimed via a collimator 420 at a beam splitter 440. The beam splitter 440 outputs a portion of the cw laser power, which is passed on via a focusing lens 460 and an optical fiber 470 to a handpiece 100′.

That portion of the cw light emitted from the laser diode 410 which is not output from the beam splitter 440 is passed on continuously via a focusing lens 430 and an optical fiber 450 to a wafer laser 480, which emits pulsed laser light L.

Both the cw light from the laser diode 410, which is output by the beam splitter 440, and the pulsed laser light L, which is emitted by the wafer laser 480, are supplied to the schematically illustrated handpiece 100′.

The cw laser light from the laser diode 410, which is output by the beam splitter 440 and is supplied via the optical fiber 470 to the handpiece 100′, is passed on from all input mirror 520 by a scanner apparatus 530 via a focusing lens 540 and a deflection mirror 550, and is passed out of the handpiece 100′, such that it can be used for treatment or diagnosis.

The pulsed laser light L, which is emitted by the wafer laser 480, is likewise passed into the handpiece 100′, and passes through the input mirror 520, which is transparent for this wavelength, and then, analogously to the above description, cw laser light from the laser diode 410, which has been output from the beam splitter, passes through the scanner 530, the focusing lens 540, and is passed on via a deflection mirror 550 out of the handpiece 100′ to the exterior, for treatment or diagnosis.

The embodiment of the medical laser treatment appliance according to the invention as shown in FIG. 3 may have a number of apparatuses by means of which the user can choose which emitted light he wishes to use for the treatment or diagnosis. By way of example, shutters 492,592,594 are provided in FIG. 3, which can be controlled by the user such that they can be used to shield or pass on the cw and/or pulsed laser light.

In the embodiment shown here, a shutter 492 is provided for possible shielding of cw laser light actually outside the handpiece 100′, while two further shutters 592, 594 are provided in the handpiece 100′. It is, of course, also possible to position the shutters 492, 592, 594 differently, for example dispensing with the shutter 492 or 592, or using other elements with a comparable effect, for example a mirror apparatus, instead of the shutters.

The user can thus control whether he wishes to use only the cw light from the laser diode 410, which is output from the beam splitter 440, or the pulsed laser light L emitted from the wafer laser 480, or else a combination of both emitted radiations. It is, of course, also possible to use the shutters to completely suppress emission of laser light from the handpiece 100′ quickly and without any problems, and without having to switch off the laser light sources per se.

The illustrated embodiment uses a diode laser 410 which emits at a wavelength of λ=809 nm and is particularly highly suitable for coagulation, while the wafer laser 480 emits at a wavelength of λ=1030 nm. A conventional Yb:KGW lasers, a Yb:KYW laser or else an Nd:Y AG laser could also be used instead of the wafer laser, emitting light at a wavelength of A=1064 nm. Other cw laser sources may also be used instead of the laser diode 410.

The user thus has the option of selecting between cw laser radiation and pumped laser radiation, and also has the option of using cw laser light and pulsed laser light superimposed at the same time.

With regard to the embodiment illustrated in FIG. 3, it should also be noted that the beam splitter 440 continuously passes on a portion of the laser light emitted from the laser diode 410 to the wafer laser 480, so that no damaging power fluctuations, or only power fluctuations which do not damage the operation of the wafer laser 480, occur in the downstream re-generative amplifier or in the wafer laser 480. In principle, it is possible to design the beam splitter 440 such that a portion of the laser light produced by the laser diode 410 is output for treatment purposes only when the light produced by it is also actually being used for treatment and diagnosis. For example, the beam splitter 440 can be arranged such that it can be moved and can be folded away when all the pump powers of the laser diode 410 are intended to be provided for the downstream laser system 480.

A number of laser diodes are preferably also used instead of one laser diode 410. In addition to the embodiment shown in FIG. 3, it is also possible, in addition, to arrange a further laser diode (or some other laser beam source) as a further first laser light source, independently of the laser diode 410, and to couple it at any desired point into the laser treatment appliance, preferably into the handpiece 100′, while the laser diode 410, as a first laser light source, is used mainly as a pump laser for the wafer laser 480 (or some other second laser light source). It is possible, by way of example, to use optical fibers as a simple means for passing on and inputting this (additional) laser light into the handpiece 100′, An embodiment such as this has the advantage that it is not absolutely essential to provide the beam splitter 440 shown in FIG. 3 although, nevertheless, this can still be provided in order to ensure a high level of variability of the overall system.

The embodiment shown in FIG. 3 furthermore has a half-wave plate 425 which can be rotated and by means of which the polarization direction of the laser light can be influenced as it passes through this half-wave plate 425. In this case, the beam splitter 440 is a beam splitter whose transmission or reflection coefficient is dependent on the polarization direction of the laser light arriving at it, so that it is possible by rotating the half-wave plate 425, which can be rotated, to determine the proportion of the cw laser light which is output through the beam splitter 440 or which is passed to the wafer laser 480. This provides a simple means for continuously variably controlling the power levels of the cw laser light which, on the one hand, are passed on continuously to the wafer laser 480 (or to some other second laser light apparatus) so that pulsed operation can be maintained, and is, on the other hand, output and is input into the handpiece 100′. It should be mentioned at this point that the half-wave plate 425, which can be rotated, and the beam splitter 440 could be aligned or configured such that virtually 100% of the cw radiation emitted from the laser diode 410 can be passed to the wafer laser 480 while, on the other hand, it is also possible to considerably reduce the “pump power” to the wafer laser 480.

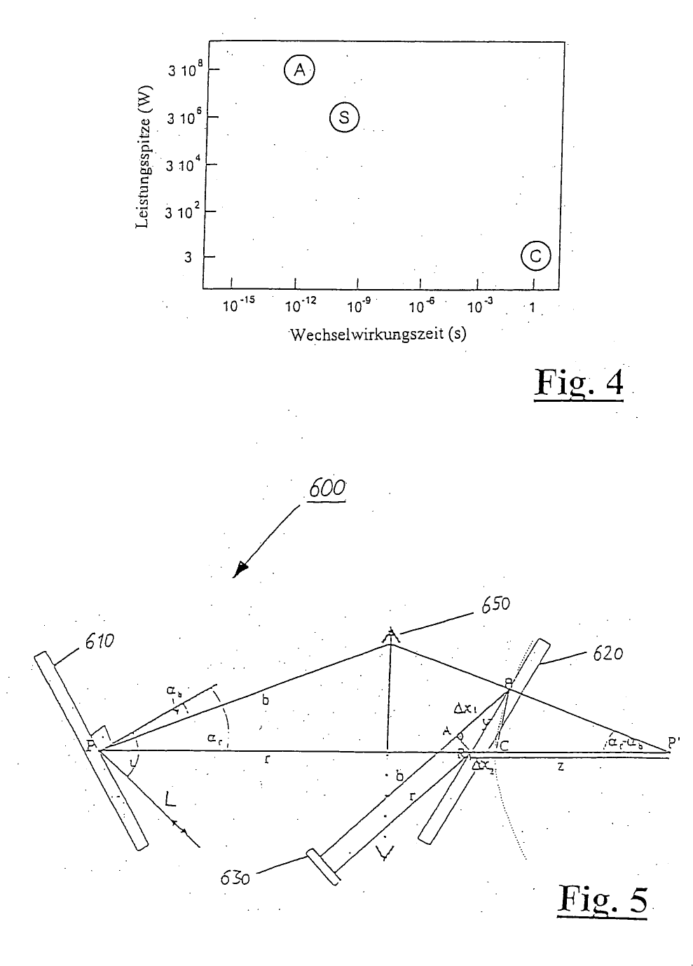

FIG. 4 shows preferred parameters for various operating states of the medical laser treatment appliance according to the invention, indicating not only the interaction time in seconds but also the power peak in the chosen operating mode.

The area annotated by “C” is a cw operating mode with a continuous power level of about 3 watts. The interaction time is in this case stated to be one second, although this need not correspond to the maximum treatment duration and, in general, it is preferable to carry out a treatment lasting for several seconds.

The areas annotated “S” and “A” each correspond to pulsed operating modes of the medical laser treatment appliance according to the invention, with a pulse energy of 300 μj being produced in the area annotated S with a pulse duration of approximately 100 ps, corresponding to a maximum pulse power of approximately 3×10⁶ W. This area is particularly suitable for sealing a hard substance, in particular in the field of dentistry.

The pulse energy in the area annotated “A” is likewise 300 μj, but this is emitted over a pulse duration of less than 1 ps, which corresponds to a peak pulse power of about 3×10⁸ W. This mode is particularly suitable for ablation of hard material, in particular for dental treatment.

Further areas and operating modes may, of course, also be used. The areas illustrated in FIG. 4 indicate merely particularly preferred operating areas of one embodiment of a medical laser treatment appliance.

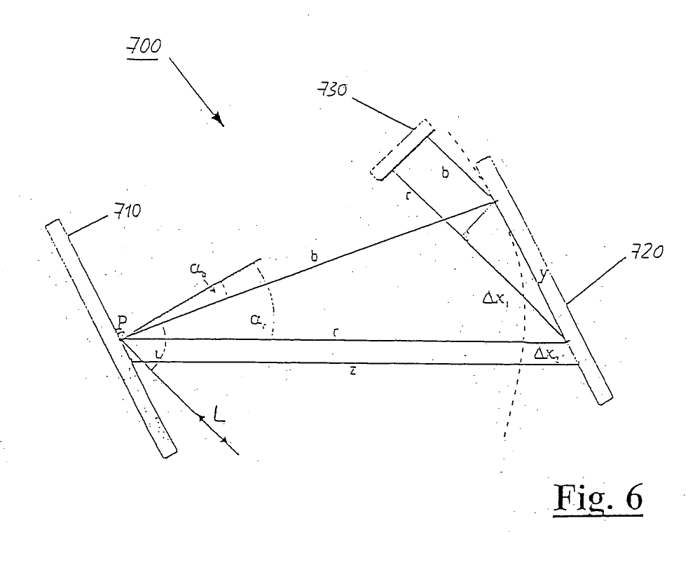

FIGS. 5 and 6 illustrate elements for varying the pulse length, as are used, for example, in conjunction with CPA method (Chirped Pulse Amplification). FIG. 5 in this case shows a stretcher 600, which lengthens the time duration of an arriving laser pulse, while FIG. 6 shows a compressor 700, which shortens or compresses the length of an arriving laser pulse.

The illustrated elements make use of the dispersive characteristics of gratings and prisms in order to draw the arriving pulses apart from one another, or to compress them.

If, as is shown in FIG. 5, a laser pulse L arrives at the angle ι at the point P on a reflection grating 610, then the spectral components of the pulse L are reflected at different angles. The beams b and r, which represent the different wavelengths, leave the grating 610 in the first order at the angles α₃ and αᵣ. In addition, a lens 650 is positioned such that the point P would be mapped 1:1 to the point P′.

In addition to the lens 650, a second grating 620 is positioned such that all the beams which are reflected on the grating 610 at a specific angle arrive at the grating 620 at this angle. Since the diffraction angle on the grating 620 is thus precisely ι for all beams, all the spectral components run parallel once again after the grating 620. The grating 620 can be moved along the path annotated Z, thus resulting in variable, different path lengths for the beams r and b after the second grating.

The different spectral components are thus subjected to different delay times, thus intrinsically resulting in the pulse beam length after reflection (chirp).

While positive chirp is produced in the stretcher 600, the chirp in the compressor 700 shown in FIG. 6 is negative. Analogously to the stretcher shown in FIG. 5, the compressor shown in FIG. 6 has a first grating 710, a second grating 720 and a mirror 730.

The pulse durations of the pulsed laser light L can thus be varied over wide ranges by moving the second grating 620 in the stretcher 600 or moving the second grating 720 in the compressor 700.

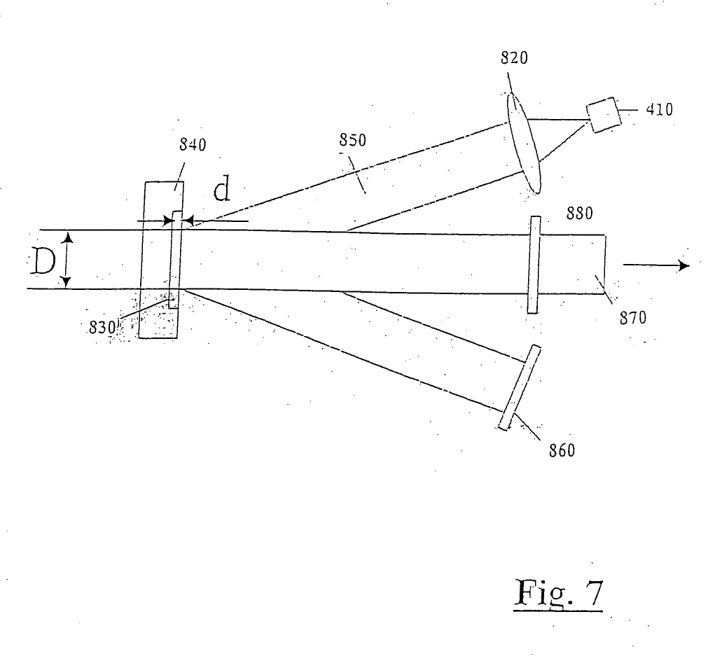

FIG. 7 shows, schematically, a cross section through a number of components of a wafer laser system, as can be used as the second laser light source in the laser treatment appliance according to the invention, with FIG. 7 also additionally showing, schematically, the elements of the first laser light system, in this case in particular the laser diode 410, which is used as the first laser light source. However, FIG. 7 shows only those elements which are required for pumping or exciting the wafer laser, while elements which are required for partial outputting of the laser power produced by the laser diode 410 are not shown in FIG. 7, in order to simplify the illustration. Furthermore, only one reflection path is also shown, in order to keep the illustration clear.

The cw laser light produced by the laser diode 410 passes through a lens 820 and then arrives at a crystal wafer 830 of the wafer laser, which is cooled from three sides by a cooling apparatus 840 for the wafer laser.

As can be seen well in FIG. 7, the diameter D of the pump laser beam 850 produced by the laser diode 410 is very much greater than the thickness d of the crystal wafer 830 of the wafer laser, thus allowing extremely effective cooling. Owing to the dimensions of the diameter D of the pump laser 850 and the thickness d of the crystal wafer 830, the heat flow may be regarded as being essentially one-dimensional, by which means the formation of a thermal lens is greatly suppressed, thus leading to the advantages described above.

Furthermore, FIG. 7 shows, schematically, that the pump laser beam 850 is reflected by means of a reflection element 860, in this case a fully reflective mirror. A laser beam 870 is output via an output mirror 880, with the output laser beam 870 being a pulsed laser beam, which can be used directly for treatment or diagnosis.

The wafer laser system shown in FIG. 7 is a Yb:KGW wafer laser, which emits at a wavelength of 1026 nm and has an emission bandwidth of approximately 15 nm. However, other materials can also be used, in particular for example a Yb:KYW wafer laser, as well as Yb:Y AG wafer lasers or Nd-doped materials, such as an Nd:glass wafer laser with an emission wavelength of approximately 1054 nm and an emission bandwidth of approximately 20 nm.

The features disclosed in the above description, in the claims and in the drawings may be significant for the implementation of the various embodiments of the invention both individually and in any combination.

1. A medical laser treatment appliance which is designed to be switchable between a cw (continuous wave) operating mode and a pulsed operating mode and has at least one first laser light source (410) for producing a cw laser beam,
characterized in that
the laser treatment appliance furthermore has a second laser light source (480) for producing a pulsed laser beam, which is pumped by at least one of the at least one first laser light sources (410), and in which cw operating mode the cw laser beam from at least one of the at least one first laser light sources (410) is used for the treatment, and, in the pulsed, operating mode, the pulsed laser beam from the second laser light source (480) is used for the treatment, with the laser treatment appliance being designed such that the second laser light source (480) is pumped continuously, even in the cw operating mode, by at least one of the at least one first laser light sources (410).

2. The medical laser treatment appliance as claimed in claim 11, characterized in that the at least one first laser light source (410′) comprises a solid state laser.

3. The medical laser treatment appliance as claimed in claim 1 or 2, characterized in that the at least one first laser light source (410) comprises at least one laser diode.

4. The medical laser treatment appliance as claimed in one of claims 1 to 3, characterized in that the second laser light source (480) comprises a wafer laser.

5. The medical laser treatment appliance as claimed in one of the preceding claims, characterized in that the second laser light source (480) is designed such that it can be switched between different pulsed operating modes, in which it emits laser light with different laser pulse durations and/or at different pulse repetition frequencies.

6. The medical laser treatment appliance as claimed in one of the preceding claims, characterized in that the second laser light source (480) is designed such that pulsed laser light can be emitted with a pulse duration t, where 20 ps≦t≦500 ps.

7. The medical laser treatment appliance as claimed in one of the preceding claims, characterized in that the second laser light source (480) is designed such that pulsed laser light can be emitted with a pulse duration t, where t≦1 ps.

8. The medical laser treatment appliance as claimed in one of the preceding claims 1 to 5, characterized in that the second laser light source (480) is designed such that pulsed laser light can be emitted with a pulse duration t, where t=5 to 500 fs.

9. The medical laser treatment appliance as claimed in one of the preceding claims, characterized in that the second laser light source (480) is designed such that pulsed laser light can be emitted with a pulse duration t, where t≧1 ns.

10. The medical laser treatment appliance as claimed in one of the preceding claims, characterized in that the appliance is designed such that light can be emitted at at least one wavelength λ, where λ is between 750 nm and 1.100 nm.

11. The medical laser treatment appliance as claimed in claim 10, characterized in that one first laser light source (410) is designed such that it emits a first wavelength λ₁ for a cw operating mode, where λ₁ is in a wavelength range from 750 nm to 1100 nm.

12. The medical laser treatment appliance as claimed in claim 10 or 11, characterized in that the second laser light source (480) is designed such that it emits a second wavelength λ₂ for a pulsed operating mode, where λ₂ is between 750 nm and 800 nm.

13. The medical laser treatment appliance as claimed in one of the preceding claims, characterized in that the second laser light source (480) comprises a Yb:KGW laser, a Yb:KYW laser, a Yb:YAG laser, an Nd:YAG laser or an Nd:YLF laser.

14. The medical laser treatment appliance as claimed in one of the preceding claims, characterized in that said appliance comprises a control apparatus and/or a switching apparatus for selecting the operating modes and/or other functions of the medical laser treatment appliance.

15. The medical laser treatment appliance as claimed in one of the preceding claims, characterized in that said appliance furthermore comprises an apparatus (340) for producing and/or controlling a visible auxiliary laser beam.

16. The medical laser treatment appliance as claimed in one of the preceding claims, characterized in that said appliance furthermore comprises at least one means for changing the pulse duration of the laser beam which is emitted from the second laser light source (480).

17. The medical laser treatment appliance as claimed in one of the preceding claims, characterized in that said appliance furthermore comprises a beam splitter (440) which outputs a portion of the emitted power of the at least one first laser light source (410).

18. The medical laser treatment appliance as claimed in one of the preceding claims, characterized in that said appliance has controllable shielding means (492, 592, 594), by means of which cw laser light from at least one first laser light source (41) and/or pulsed laser light from the second laser light source (480) can be shielded.
light source and wherein in the continuous wave operating mode said continuous wave laser beam from at least one of said at least one first laser light sources is used for treatment and in the pulsed operating mode the pulsed laser beam from said second laser source is used for treatment and wherein said laser treatment appliance in operation has said second laser light source pumped continuously even in the continuous operating mode by at least one of said at least one first laser light sources.

20. The medical laser treatment appliance as claimed in claim 19 wherein said at least one first laser light source comprises a solid state laser.

21. The medical laser appliance as claimed in claim 19 wherein said at least one first laser light source comprises at least one laser diode.

22. The medical laser treatment appliance as claimed in claim 19 wherein said second laser light source comprises a wofer laser.

23. The medial laser treatment appliance as claimed in claim 19 wherein said second laser light source can be switched between different pulse duration and/or at different pulse repetition frequencies.

24. The medical laser treatment appliance as claimed in claim 19 wherein said second laser light source is pulsed at different repetition frequencies with a pulse duration t, where ps≦t≦500 ps.

25. The medical laser treatment appliance as claimed in claim 19 wherein said second laser light source is pulsed at different repetition frequencies with a pulse duration t, where t≦1 ps.

26. The medical laser treatment appliance as claimed in claim 19 wherein said second laser light source is pulsed at different repetition frequencies with a pulse duration t, where t=5 to 500 fs.

27. The medical laser treatment appliance as claimed in claim 19 wherein said second laser light source is pulsed at different repetition frequencies with a pulse duration t, where t≧1 ps.

28. The medical laser treatment appliance as claim 19 wherein the laser light may be emitted at least one wavelength λ where λ is between 750 nm and 100 nm.

29. The medical laser treatment apparatus as claimed in claim 28 wherein said first laser light source emits laser light at a first wavelength λ for a continuos operating mode wherein λ₁ is a wavelength range from 750 nm to 1100 nm.

30. The medical laser treatment appliance as claimed in claim 28 wherein said second laser light source emits a second wavelength λ₂ for a pulsed operating mode wherein λ₂ is between 750 nm and 800 nm.
